(19) United States
(12) Patent Application Publication
(10) Pub. No.: US 2012/0259153 A1
(43) Pub. Date: Oct. 11, 2012

(54) DELIVERY SYSTEMS FOR BRACHYTHERAPY, AND ASSOCIATED METHODS

(75) Inventors: Zhuang Wang, Durham, NC (US); Michael Edward Werner, Morrisville, NC (US); James Donald Byrne, Carrboro, NC (US); Ronald Chingyun Chen, Chapel Hill, NC (US); Shrirang Shrikant Karve, Chapel Hill, NC (US); Joseph DeSimone, Chapel Hill, NC (US)

(73) Assignee: THE UNIVERSITY OF NORTH CAROLINA AT CHAPEL HILL, Chapel Hill, NC (US)

(21) Appl. No.: 13/502,949

(22) PCT Filed: Oct. 20, 2010
(86) PCT No.: PCT/US10/53357

§ 371 Date: Jun. 25, 2012

Related U.S. Application Data
(60) Provisional application No. 61/253,547, filed on Oct. 21, 2009.

(51) Int. Cl. A61M 36/12; B82Y 5/00

(52) U.S. Cl. 600/8; 977/773; 977/906

(57) ABSTRACT

Delivery systems adapted for implementation during a brachytherapy procedure are provided. The delivery system includes a plurality of brachytherapy seeds and a plurality of spacers. Each spacer is formed of a matrix material carrying a plurality of microparticles and/or nanoparticles. The microparticles and/or nanoparticles carry an agent and are biodegradable and/or biocompatible. The brachytherapy seeds and the spacers are configured to be delivered to a target site. Associated methods are also provided.

### BACKGROUND

1.Field of the Invention

Embodiments of the present invention relate to delivery systems, and more particularly, to delivery systems and associated methods for facilitating delivery of various agents to sites targeted during a brachytherapy procedure.

2. Description of Related Art

Many techniques exist for the delivery of drugs and therapeutic agents to the body. Traditional delivery methods include, for example, oral administration, topical administration, intravenous administration, and intramuscular, intradermal, and subcutaneous injections. With the exception of topical administration which permits more localized delivery of therapeutic agents to a particular area of the body, the aforementioned drug delivery methods generally result in systemic delivery of the therapeutic agent throughout the body. Accordingly, these delivery methods are not appropriate for localized targeting of drugs and therapeutic agents to specific internal body tissues.

For example, brachytherapy requires placement of radioactive seeds into a target site such as the prostate gland. While most brachytherapy procedures involve the permanent placement of radioactive seeds into the tissue gland for radiation delivery over a substantial period of time (termed low dose rate brachytherapy or LDR), some brachytherapy procedures involve what is termed as high dose rate brachytherapy (or HDR). HDR involves the placement of hollow plastic catheters (typically introduced inside hollow stainless steel needles) into tissue, followed by the “afterloading” of a radiation source (e.g., Ir-192) into these catheters for a defined period of time. After radiation is delivered by the HDR source from variable positions within these catheters to tissue as per the computer-generated plan for appropriate radiation dose delivery, the radiation source is retracted from target tissue and the catheters are removed.

However, brachytherapy seed implant techniques cause inflammation of the tissue surrounding the tract. One example is the inflammation of the prostate gland secondary to the intrinsically damaging effects of radiation on tissue. However initial swelling of the gland is due to the brachytherapy needle insertion into the prostate gland. Inflammation, swelling, and subsequent related symptoms and long term effects from prostate seed implant are due to both the procedural trauma and the radiation effects of the seeds.

The placement and total number of radioactive seeds is designed to achieve proper dose distribution within the pre-implant gland volume. The procedural swelling immediately after the implant can result in the seeds separating a small degree, affecting the overall dose distribution. The procedurally-induced enlargement of the prostate therefore, in most patients, results in the seeds being further apart from each other until this swelling resolves. The planning of seed placement cannot predict accurately such post-implant prostate volume enlargement. Since the degree of enlargement of the prostate gland cannot be predicted and can be quite variable, no effort is made to “guess” on the volume changes. Dose prescriptions have taken into account this enlargement but since not all glands swell proportionately the same amount, it would be desirable to minimize the gland enlargement particularly during the first thirty days when the majority of the enlargement occurs. While local cancer control rates have not yet been affected by this enlargement, the potential for high dose and low dose regions remains and these high dose regions likely have significant effect on the acute and chronic side effects.

A more acute impact of the brachytherapy for prostate gland enlargement and swelling after seed implant is the acute patient morbidity relating to urinary difficulty (obstruction) and discomfort. All patients experience varying degrees of urination related morbidity related to the procedure. Most patients experience some pain upon initial urinations after the procedure. Approximately 5-10% of patients require a temporary catheter and, in rare circumstances, a catheter for a period of many months. The immediate effects are related to the immediate procedural induced enlargement of the gland and, in the ensuing months, effects by the radiation induced swelling within the gland. Patients may experience the radiation-induced swelling as a slowing of the urinary stream, slight pain, urgency, or frequency. These symptoms are caused by compression of the urethra. Compression of the urethra by internal prostate swelling can cause, in more extreme situations, complete urinary obstruction. If medication cannot keep the passage adequately unobstructed after seed implantation, then the patient will require Foley catheterization, daily self-catheterization, or suprapubic diverting cystostomy. Persistent swelling and obstruction may ultimately result in eventual procedural intervention. While these obstructive and irritative symptoms may occur as the primary morbidities following prostate seed implant, lesser but relevant problems include rectal morbidities and sexual dysfunction related to the implant procedure.

Accordingly, it would be desirable to provide an improved apparatus and method for selectively and locally targeting delivery of various agents during a brachytherapy procedure so as to decrease the side effects of brachytherapy. Furthermore, the main goal of brachytherapy is the treatment of cancer in the target tissue. There are a wide array of agents that can be given together with radiotherapy to improve the efficacy of treatment. Such agents include chemotherapy, biologics, siRNA and they have been demonstrated in preclinical and clinical studies. A method to incorporate various drugs into brachytherapy can also improve the efficacy of the treatment. In the case of prostate brachytherapy, it would be desirable to provide an apparatus and method capable of delivery and sustained release of anti-inflammatory drugs and chemotherapy agents, wherein the anti-inflammatory drugs may improve the response of the tissue to inflammation, and the delivery of other agents may improve the effectiveness of the brachytherapy system. This system can also be applied to brachytherapy for any tumor site, and is not limited to prostate cancer. Brachytherapy would allow selective and targeted delivery of radiation therapy and/or specified agents to any tumor reachable by the brachytherapy method.

### SUMMARY

The present invention relates to a delivery system and method, and in particular, a delivery system adapted for implementation during a brachytherapy procedure. The delivery system comprises a plurality of brachytherapy seeds. An insertion device is configured to receive the brachytherapy seeds. A plurality of spacers is interspersed between the brachytherapy seeds within the insertion device. Each spacer comprises a degradable or biocompatible matrix material interspersed with microparticles and/or nanoparticles carrying an agent or many agents. The microparticles and nanoparticles are biodegradable and/or biocompatible. The insertion device is configured to deliver the brachytherapy seeds and the spacers to a target site.

Other aspects of the present invention relate to methods for co-delivery of an agent during a brachytherapy procedure. The method includes delivering a plurality of brachytherapy seeds proximate to a target site. The method further comprises delivering a plurality of spacers proximate to the target site, wherein the spacers are interspersed between the brachytherapy seeds, and each spacer comprises a degradable matrix material interspersed with microparticles and/or nanoparticles carrying an agent. The microparticles and nanoparticles are biodegradable and/or biocompatible.

According to other aspects of the present invention, a delivery system adapted for implementation during a brachytherapy procedure, wherein the delivery system comprises an elongate tubing capable of insertion into a bodily orifice. The tubing defines a lumen extending therethough such that an agent is capable of being transported through the tubing. A first electrode is operably engaged with the elongate tubing such that the first electrode is capable of being positioned proximate to a target site. A second electrode is in electrical communication with the first electrode and is opposably positionable with respect thereto such that the target site is at least partially disposed therebetween. The second electrode is configured to cooperate with the first electrode to form an electric field for directing the agent transported through the tubing toward the target site during interaction therebetween.

Other aspects of the present invention provide a method for delivering an agent to a target site. Such a method comprises extending an elongate tubing within a bodily orifice such that a first electrode operably engaged with the tubing is disposed proximate to a target site, wherein the elongate tubing defines a lumen extending therethrough. The method further comprises opposably positioning a second electrode with respect to first electrode such that the target site is disposed at least partially between the first and second electrodes. The method further comprises applying a voltage potential across the first and second electrodes to form an electric field, and supplying an agent proximate to the target site via the lumen such that the agent interacts with the electric field so as to be directed toward the target site.

Particles can be pico, nano and microparticles. In certain embodiments, the particle is a polymeric particle. In certain embodiments, the particle comprises a polymeric core with a shell coating the core. In certain embodiments, the particle comprises a polymeric core with a lipid coating. In certain embodiments, the particle is a liposome. In certain embodiments, the particle is a micelle.

The whole particle or a portion of the particle may be biodegradable. The spacer or part of the spacer maybe biodegradable.

In certain embodiments, a particle is any entity having a greatest dimension of less than 500 microns. In certain embodiments, an particle is any entity having a greatest dimension of less than 300 microns. In certain embodiments, an particle is any entity having a greatest dimension of less than 200 microns. In certain embodiments, an particle is any entity having a greatest dimension of less than 100 microns. In certain embodiments, an particle is any entity having a greatest dimension of less than 75 microns. In certain embodiments, an particle is any entity having a greatest dimension of less than 50 microns. In certain embodiments, an particle is any entity having a greatest dimension of less than 10 microns. In certain embodiments, an particle is any entity having a greatest dimension of less than 1000 nm. In certain embodiments, an particle is any entity having a greatest dimension of less than 900 nm, 800 nm, 700 nm, 600 nm, 500 nm, 400 nm, 300 nm, 200 nm, 100 nm, or 10 nm.

In some embodiments, the particles are spheres, spheroids, flat, plat-shaped, cubes, cuboids, ovals, ellipses, cylinders, cones, or pyramids.

In some embodiments, the particle and/or the spacer can be composed of polyethylenes, polycarbonates, polyanhydrides, polyhydroxyacids, polypropylfumerates, polycaprolactones, polyamids, polyacetals, polyethers, polyesters, polyorthoesters, polycyanoacrylates, polyvinyl alcohols, polyurethanes, polyphosphazenes, polyacrylates, polymethacrylates, polyureas, polystyrenes, polyamines, proteins, lipids, surfactants, carbohydrates, small molecules, and/or polynucleotides.

In another embodiment, a delivery system adapted for implementation during a brachytherapy procedure is provided. The delivery system includes a plurality of brachytherapy seeds and a plurality of spacers, wherein each spacer includes a matrix material carrying a plurality of microparticles and/or nanoparticles. The microparticles and/or nanoparticles carry at least one agent and are biodegradable and/or biocompatible. The brachytherapy seeds and the spacers are configured to be delivered to a target site.

According to various aspects of the delivery system, each spacer comprises a matrix material mixed with the microparticles and/or nanoparticles. Alternatively, each spacer includes a matrix material housing the microparticles and/or nanoparticles. In one embodiment, the microparticles and/or nanoparticles are biodegradable. The matrix material may also be a biodegradable material. The microparticles and/or nanoparticles can be various sizes, such as about 10 nm to 500 μm in diameter (e.g., 230-250 nm in diameter). In addition, the microparticles and/or nanoparticles may be fabricated using different techniques such as, for example, a soft or an imprint lithography technique. The microparticles and/or nanoparticles can be various types of materials such as poly(lactic-co-glycolic acid), polylactic acid, polyglycolic acid, chitosan, lipids, and/or poly(R-aminoester).

In additional aspects, the agent comprises anti-inflammatory agents (e.g. but not limited to, dexamethasone, ibuprofen, prednisone, betamethasone, or glucocorticoid), and/or anti-androgen agents (e.g., bicalutamide, flutamide, or nilutamide), and/or chemotherapy agents (e.g., docetaxel, paclitaxel, cisplatin, gemcitabine, or doxorubicin), and/or agents that improve urination (e.g., terazosin or tamsulosin), and/or genetic agents, and/or biologics, and/or siRNA, and/or imaging agents, and/or protein agents, and/or molecular targeted agents against cancer. The agent may be encapsulated by at least one of the nanoparticles or microparticles. The agent may be configured to be gradually released over time. Moreover, each of the brachytherapy seeds may include a radioactive isotope, while the matrix material may include a plastic, a synthetic biodegradable polymer, and/or a natural polysacchride.

In one aspect, the brachytherapy seeds and/or spacers are arranged in an alternating pattern such that each seed is separated by a spacer. The plurality of seeds and spacers may be coupled to one another. The delivery system may further include an insertion device, such as a needle, configured to receive each of the brachytherapy seeds and the plurality of spacers and deliver the seeds and spacers to the target site.

According to another embodiment, a method for co-delivery of an agent during a brachytherapy procedure is provided. The method includes delivering a plurality of brachytherapy seeds proximate to a target site and delivering a plurality of spacers proximate to the target site, wherein each spacer includes a matrix material carried by a plurality of microparticles and/or nanoparticles. The microparticles and/or nanoparticles carry at least one agent and are biodegradable and/or biocompatible.

Aspects of the method include collectively or independently delivering the brachytherapy seeds and the spacers. The brachytherapy seeds and the spacers may be delivered while being coupled to one another. Furthermore, the brachytherapy seeds and the spacers may be delivered in an alternating pattern. In one aspect, the brachytherapy seeds and the spacers are delivered with an insertion device. In addition, the agent may be gradually released over time. The brachytherapy seeds and the spacers may be delivered to different target sites such as into a prostate gland.

As such, embodiments of the present invention are provided to enable a highly targeted and efficient delivery of an agent to predetermined target sites.

### BRIEF DESCRIPTION OF THE DRAWINGS

Having thus described various embodiments of the invention in general terms, reference will now be made to accompanying drawings, which are not necessarily drawn to scale, and wherein:

FIG. 1 illustrates a brachytherapy procedure employing ultrasound-image guidance;

FIG. 2 illustrates a brachytherapy procedure;

FIG. 3 illustrates an insertion device used during a brachytherapy procedure, according to one embodiment of the present disclosure;

FIG. 4 illustrates another insertion device used during a brachytherapy procedure, according to one embodiment of the present disclosure;

FIG. 5 illustrates yet another insertion device used during a brachytherapy procedure, according to one embodiment of the present disclosure;

FIG. 6 illustrates brachytherapy seeds separated by spacers dispersed within a prostate gland;

FIGS. 7A-7C illustrate views of a spacer implemented during a brachytherapy procedure, according to various aspects of the present disclosure;

FIG. 8 illustrates a spacer having particles carrying an agent, the spacer being interspersed between a pair of brachytherapy seeds, according to one aspect of the present disclosure;

FIGS. 9A and 9B illustrate various partial views of an insertion device having an electrode in communication therewith, according to one embodiment of the present disclosure;

FIG. 10 illustrates an electrode configured for use during a brachytherapy procedure, the electrode having a matrix material carrying an agent, according to one embodiment of the present disclosure;

FIGS. 11A and 11B illustrate various views of a delivery system configured to deliver an agent to a target site, according to one embodiment of the present disclosure;

FIG. 12 illustrates implementation of the delivery system of FIGS. 11A and 11B to deliver an agent to at least one target site, according to one embodiment of the present disclosure;

FIGS. 13 and 14 are graphs illustrating exemplary results from experimental testing according to one embodiment of the present disclosure;

FIGS. 15 and 16 are images of exemplary spacers resulting from experimental testing according to embodiments of the present disclosure; and

FIG. 17 is an image of an exemplary spacer resulting from experimental testing according to one embodiment of the present disclosure.

### DETAILED DESCRIPTION OF THE PREFERRED EMBODIMENTS

Embodiments of the present inventions now will be described more fully hereinafter with reference to the accompanying drawings. The invention may be embodied in many different forms and should not be construed as limited to the embodiments set forth herein; rather, these embodiments are provided so that this disclosure will satisfy applicable legal requirements. Like numbers refer to like elements throughout.

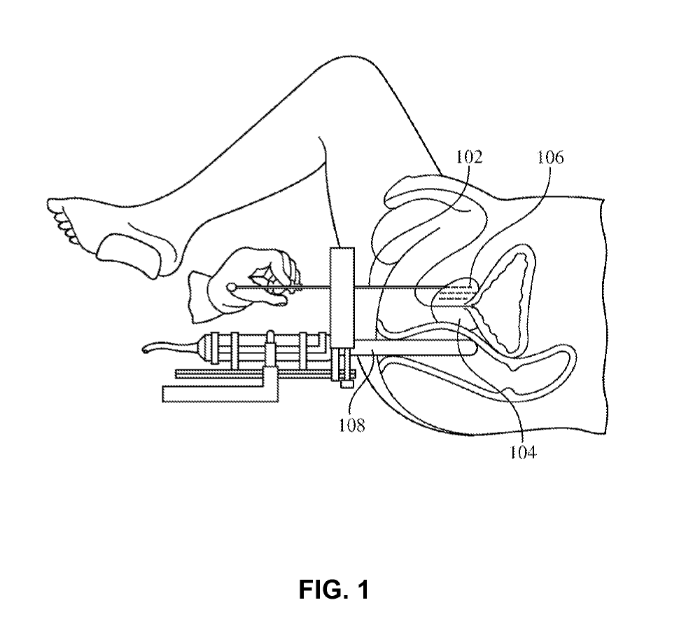

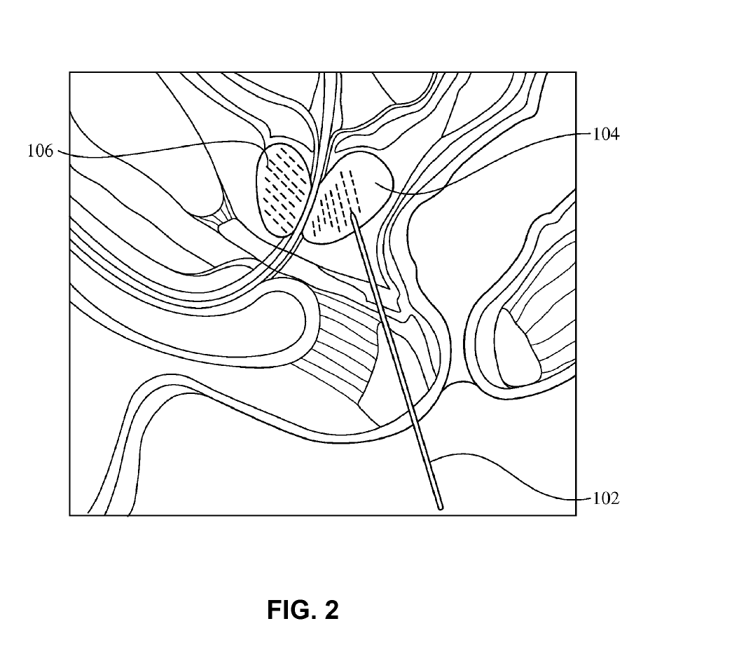

Brachytherapy refers to a localized method of treating cancer that places radioactive sources directly within tissue. The advantage of brachytherapy is that very high doses of ionizing radiation are delivered to a localized area such that the radiation is supplied primarily to the treatment area without significantly affecting tissues throughout the body. This ability, when combined with a rapid reduction in the radiation dose as a function of distance, shields distant anatomies from unwanted radiation. Hence, the technique may provide excellent results for localized treatment of various tumors. Prostate brachytherapy (seed implantation) is generally a simple one hour outpatient procedural procedure involving the insertion of usually 20-30 hollow needles 102 (catheters) through the perineal skin and into the prostate gland 104 to deliver radioactive brachytherapy seeds 106 thereto, as shown in FIGS. 1 and 2. Rectal ultrasound guidance (rectal probe 108) or other imaging modalities (e.g., MRI guidance) may be used to guide the needles.

Placement of brachytherapy seeds requires carefully planning. The procedure may be pre-planned prior to the procedure or intraoperatively, during the procedure. Both techniques require a detailed rectal ultrasound mapping outlining the shape and size of the prostate. Using such volumetric data of the gland and knowledge of the required dose, a radiation physicist determines the number of seeds and location of seed placement within the prostate gland required to deliver the prescribed dose. The prostate gland and a small amount of surrounding tissue are included in this treatment volume.

After the implant, a CT scan may be performed and calculations made to determine the actual dose and quality of the implant.

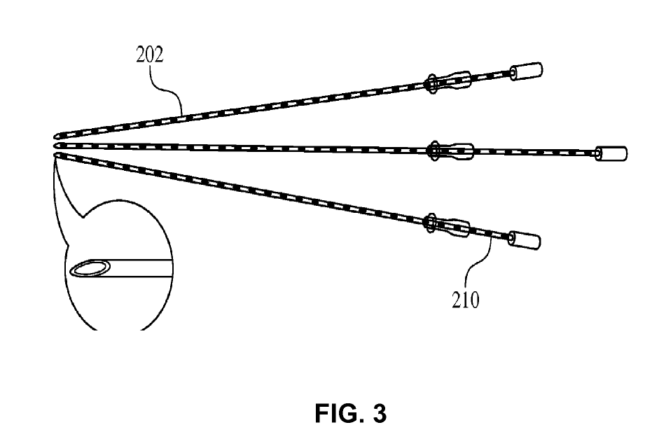

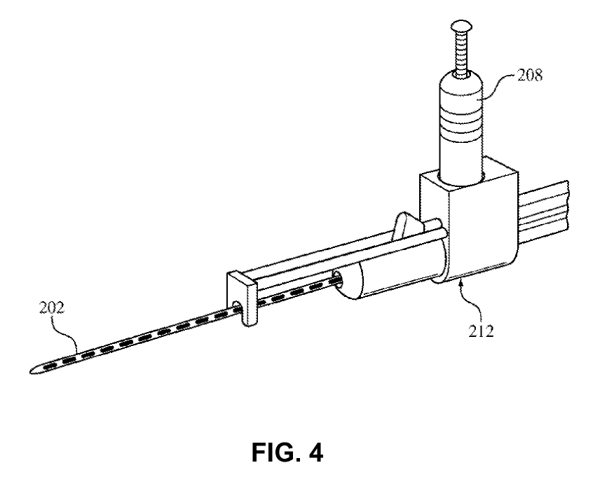

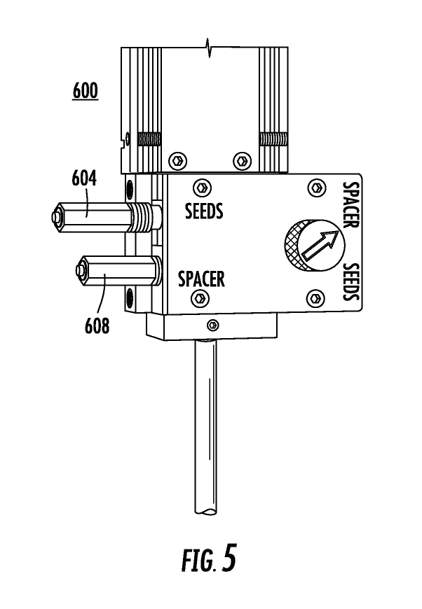

During the procedure, radioactive brachytherapy seeds are introduced into the prostate via needles. These needles may be preloaded with seeds or alternatively loaded after the needle is placed. Introduction of the seeds/needles into the prostate may be accomplished suing various insertion devices. For example, a Mick afterloading system may be used to introduce the seeds into the prostate, wherein the radioactive seeds may be released individually into the prostate gland after a trocar and sleeve are placed into the target. As shown in FIG. 3, the Mick afterloading system may include needles 202 through which seeds are delivered. Further, the Mick afterloading system may include a seed cartridge, as shown in FIG. 4. An example of a Mick afterloading system is disclosed in U.S. Patent Application Publication No. 2007/0225544 A1, which is hereby incorporated by reference herein in its entirety. As shown in FIG. 5, an insertion device 600 may include a dual chamber modification for a Mick applicator system wherein the insertion device includes one chamber 604 that houses the radioactive seeds and a second chamber that holds “spacers” 608. In other instances, an insertion device may be used such as that disclosed in U.S. Patent Application Publication No. 2006/0063961 A1 to Drobnik et al., which is hereby incorporated by reference in its entirety.

With either preloaded or the Mick System, the needle and subsequent seed placement follows a pre or intraoperative plan. The needle and seed placement may be facilitated under ultrasound guidance, such that relatively equal spacing and distribution of seeds is achieved and the plan requirements met. In this manner, a relatively homogenous radiation dose distribution covering the entire prostate gland may be achieved. Preloaded needle systems have an advantage over the Mick system as they allow for multiple, connected seeds to be placed into the prostate gland with each needle. In some instances, the radioactive seeds may include iodine-125 as the radioactive isotope, and, in other instances, palladium-103 may be the radioactive isotope. Typically, between 60 and 120 seeds may be inserted into a cancerous gland via 20-30 needles.

Preloaded needles may incorporate connected seed technology. In other instances, the needles may be loaded with independent or free seeds that are not physically connected. These seeds may migrate short distances in the prostate and further, if placed into the veins surrounding the gland. In this regard, the use of connected seeds has reduced this effect and improved overall dosimetry. With either free or connected seeds, multiple seeds can be placed incorrectly or migrate slightly resulting in an inhomogeneous dose distribution within the prostate gland. This can potentially result in underdosing cancer cells or alternatively overdosing normal tissue.

Real-time loading can also be accomplished using loose seeds, loose spacers and needles. A plan can be generated during a procedure, and needles are loaded based on the optimal dosimetry. Real-time planning allows adaptive treatment and modification of the plan based on the actual placement of seeds.

Underdosed regions may result in recurrent cancer spread (metastasis), and patient death. An underdosed region of prostate seed implant is typically recognized with post implant CT dosimetry and is retreated with reimplantation of additional seeds or, rarely with an additional course of external beam radiation. Overdosing may also occur. If the seeds bunch too close together and overdose critical structures such as the rectum, bladder neck, urethra, or peri-prostatic neurovascular bundles, unwanted morbidities such as radiation proctitis or tenesmus, radiation urethritis (causing dysuria and/or hematuria), bladder neck spasticity (causing urgency, incontinence, etc.), prostatitis, or impotence may result.

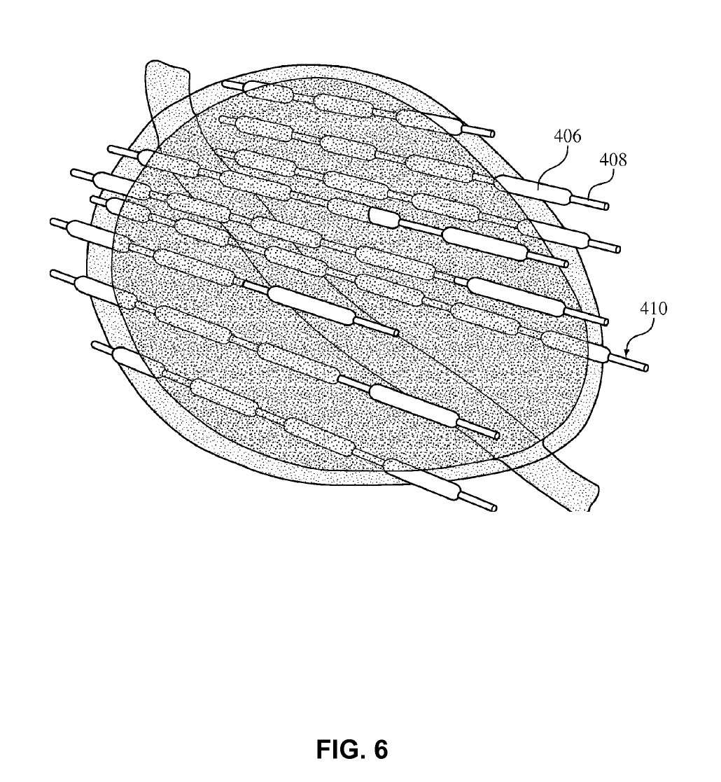

Various techniques may be used in prostate seed implants to ensure homogeneous and adequate seed number and placement location/distribution. To reduce the risk of seed migration (causing inhomogeneous, underdosed or overdosed areas within the cancerous prostate), a system of inserting an entire group of connected radioactive seeds as a “needle” unit has been introduced, as shown in FIG. 6. These “seed links or strands” 410 consist of about 2-6 radioactive seeds 406 each precisely spaced apart but connected by an inert biodegradable/absorbable non-radioactive material 408. The connected seed string thus formed creates an alternating seed, “spacer”, seed, etc. “string of pearl” pattern that ensures the correct physical separation, spatial positioning and stability of the radioactive seeds within the prostate gland for homogenous radiation dose delivery. The “spacers” are biologically inert, bio-absorbable and dissolve with time, and pose no harm or damage to the patient. As shown in FIG. 6, a biodegradable matrix “sheath” may “weld” an entire array of radioactive seeds together in a fixed “string” with appropriate separation spacing between each radioactive seed (e.g., RAPID STRAND).

Inserting the needle with preloaded, connected seeds may shorten the overall procedural time and may allow for more reliable positioning of seeds, since the linear “string of pearls” arrangement of “spacers” between consecutive radioactive seeds limits seed migration, and thus may improve accuracy of planning and homogeneity of dose delivery to the prostate. Pre-loaded needle techniques may be typically employed with pre-planned implants because flexibility of seed adjustment with pre-loaded needles during the brachytherapy procedure.

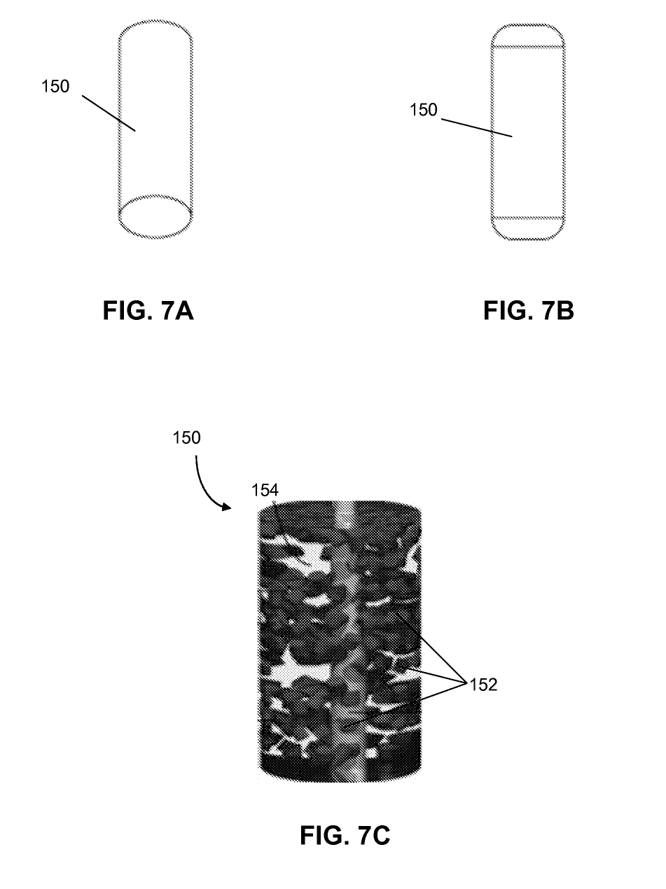

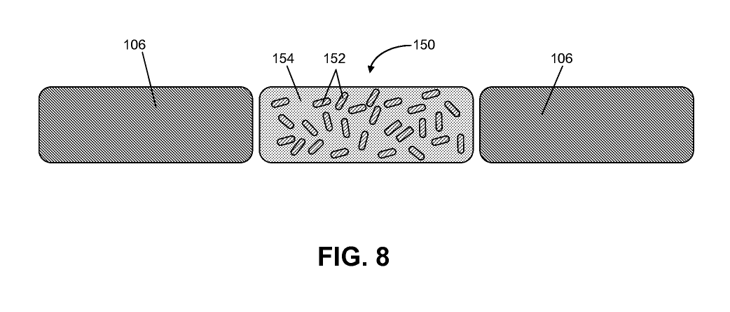

In other instances, as shown in FIGS. 7 and 8, spatial separation of the radioactive brachytherapy seeds 106 may be achieved by the use of intervening non-radioactive material (known as “spacers” 150) which may either be simply placed in a loose single-file pattern in between the radioactive seeds or otherwise physically fixed to each other and the seeds 106 by a surrounding polymeric material.

The use of multiple needles breaking through the skin and tissue and leaving behind foreign objects can lead to adverse reactions and trauma to the surrounding area. Control of trauma, bleeding and swelling during and after a brachytherapy implant procedure has long been a goal of practitioners. For example, swelling of the prostate and surrounding tissue can displace the seeds and lead to uncontrolled doses of radiation, which can involve some zones that are unintentionally hot and some that are undesirably, and possibly dangerously, cold. Trauma and swelling are commonly treated by administering systemic drugs to the patient. This has the disadvantage of requiring enough drug to be dispersed throughout the body, even though the trauma and swelling are localized. This can delay the effectiveness of the drugs.

Accordingly, embodiments of the present disclosure provide a delivery system capable of delivering spacers 150 carrying an agent for sustained delivery thereof in accordance with the present invention. In some instances, the delivery system may facilitate co-delivery of radioactive seeds 106 and agents used during a brachytherapy procedure, wherein the agent is carried to a target site using biocompatible and/or biodegradable nanoparticles and/or microparticles 152 interspersed with or otherwise carried by a matrix material 154 of the spacers 150. In one embodiment, the microparticles and/or nanoparticles are mixed together or integrated with the matrix material. However, the microparticles and/or nanoparticles could alternatively be housed by the matrix material (e.g., where the spacer is a hollow member configured to receive the microparticles and/or nanoparticles therein). The agents may be but are not limited to: anti-inflammatory drugs, anti-androgen drugs, anti-cancer drugs/chemotherapy, and medications that improve urination, imaging agents, siRNA and biologics. The agents may also be a genetic material or alpha blockers or alpha-adrenergic antagonists, as well as protein agents and molecular targeted agents against cancer. In some instances, the agents may be encapsulated by nanoparticles and/or microparticles 152 and released over time. The nanoparticles and microparticles may provide sustained drug delivery from days to months. Sustained delivery from the microparticles and nanoparticles advantageously reduces the requirement for corticosteroid injections and subsequent chemotherapy treatments.

Accordingly, the delivery system may include an insertion device for delivering the brachytherapy seeds and spacers to a site targeted for treatment. The spacers may be particularly configured to provide concurrent agent (e.g., drug) delivery during a brachytherapy procedure such as, for example, prostate brachytherapy. In some instances, the delivery system may further include an ultrasound device that guides the seeds placement, a localization device that identifies the coordinates of seed placement, and needles for seed placements.

According to one embodiment, the delivery system may include a combination brachytherapy seed and drug delivery system which includes an insertion device having a needle with a central channel, a plurality of brachytherapy seeds disposed within the central channel and a plurality of spacers containing biodegradable particles with a controlled amount of agent (e.g., drug) encapsulated therein disposed within the central channel, wherein the spacers are interspersed or otherwise positioned between the brachytherapy seeds. For example, the spacers and seeds may be arranged in an alternating pattern such that each seed is separated by a spacer. In some instances, the spacers may be cylindrical in shape, having a diameter of approximately about 10 nm to 500 microns, although other shapes may be used (e.g., spheres, spheroids, flat, plat-shaped, cubes, cuboids, ovals, ellipses, cones, or pyramids). The spacers may be composed of non-absorbable and/or non-degradable matrix material such as, for example, plastic, synthetic biodegradable polymers, natural polysacchrides, etc. In other instances, the spacers may comprise a degradable matrix material interspersed with at least one of a plurality of microparticles and nanoparticles carrying the agent.

In some instances, the agent may be encapsulated within the particles. The agent may include anti-inflammatory drugs such as but not limited to dexamethasone, ibuprofen, prednisone, betamethasone, and other glucocorticoids. The agent may also be but not limited to terazosin, tamsulosin and other medications that improves urination. The agent may also be chemotherapy agents such as but not limited to docetaxel, paclitaxel, cisplatin, gemcitabine, or doxorubicin. The agent may further be anti-androgen medications such as but not limited to bicalutamide, flutamide, and nilutamide. In addition, the agent may be a genetic material such as DNA, RNA, or sRNA, or alpha blockers or alpha-adrenergic antagonists. The particles may be sizes ranging from nanometers to micrometers in multiple dimensions. The matrix material may be configured to readily degrade, leaving degradation components, free agent (e.g., drug), and particles containing agent (e.g., drug). The particles will be composed of biodegradable materials and/or biocompatible materials, which may include, but is not limited to, poly(lactic-co-glycolic acid) (PLGA), polylactic acid (PLA), polyglycolic acid (PGA), chitosan, lipids, and poly(R-aminoester), polyethylenes, polycarbonates, polyanhydrides, polyhydroxyacids, polypropylfumerates, polycaprolactones, polyamids, polyacetals, polyethers, polyesters, polyorthoesters, polycyanoacrylates, polyvinyl alcohols, polyurethanes, polyphosphazenes, polyacrylates, polymethacrylates, polyureas, polystyrenes, polyamines, proteins, lipids, surfactants, carbohydrates, small molecules, and/or polynucleotides. The spacers may contain particles made by various particle fabrication techniques including but not limited to bottom-down approaches of solvent emulsification and self-assembly and top-down approaches including the Particle Replication In Nonwetting Templates (PRINT) technique that utilizes templates for soft or imprint lithography techniques, as disclosed in U.S. Patent Application Publication No. 2009/0028910 to DeSimone et al., filed Dec. 20, 2004, which is incorporated herein by reference in its entirety. Further, the agent may be incorporated into the matrix of the particles.

According to other embodiments, the delivery system may provide for delivering a supplemental agent to, or through, a localized area of a passageway in order to treat the localized area of the passageway or to treat a localized area of tissue located adjacent to the passageway, with minimal undesirable effect on other body tissue. Such a delivery system may be inserted intraluminally, through natural orifices, ex vivo or via direct injection. In some instances, the delivery system may include a degradable delivery component for releasing the agent in the localized area. In some instances, the delivery system may include a delivery component that may be electrochemically degraded upon the flow of a current, thereby releasing the agent to either diffuse into the surrounding tissue or, upon further application of an electric field, a charged agent (i.e., an agent being ionically charged) may be driven into the surrounding tissue by ionotophoretic techniques.

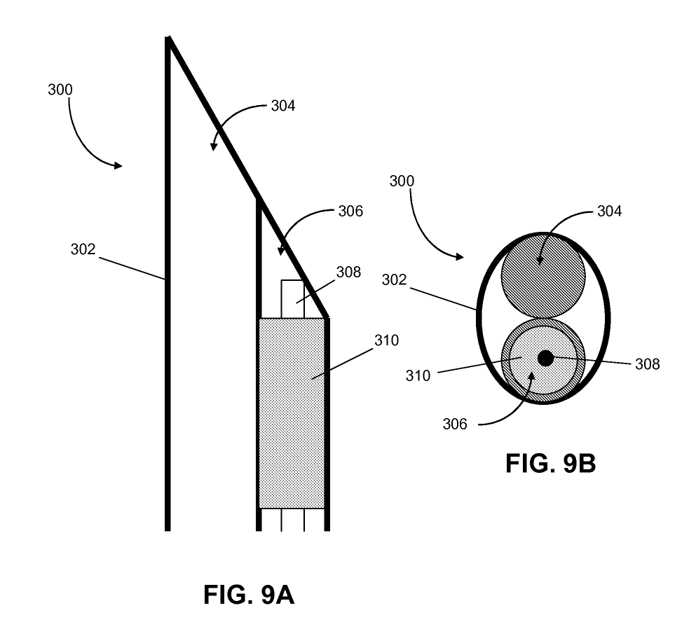

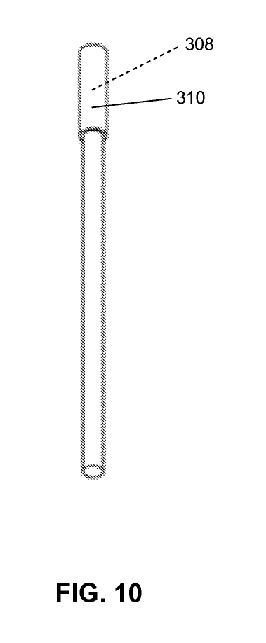

For example, as shown in FIGS. 9 and 10, the delivery system may include an insertion device 300 having a needle 302 with a first and second discharge lumen 304, 306 disposed at a distal end thereof, wherein the distal end may be positioned proximate a target site for treatment thereof. The needle may include a first and second bore disposed at a proximal end thereof. The first and second bores may extend from the proximal end to the distal end to form a central channel within the needle. Brachytherapy seeds and spacers may be delivered to the target site through the first bore and the first discharge lumen. A first (e.g., source) electrode 308 may extend within the needle through the second bore and the second discharge lumen for positioning proximate the target site. The first electrode may carry a supplemental agent (which may be the same as the agent(s) delivered by the particles interspersed within the spacer or may be an entirely different agent(s)) via a delivery component 310 disposed thereon or otherwise attached thereto, as shown in FIG. 10. A second (e.g., counter) electrode may be disposed in electrical communication with the first electrode and opposably positioned with respect thereto such that the target site is at least partially disposed therebetween. The second electrode may cooperate with the first electrode to form an electric field for directing the supplemental agent toward the target site. In some instances, one of the electrodes may be a skin patch disposed on the skin of the patient. Of course, in some instances, a single channel may be used within the needle, wherein the brachytherapy seeds/spacers and the first electrode may be delivered proximate to the target site via the single channel, either concurrently or in turn.

The delivery component 310 may, in some instances, be constructed of a degradable structure capable of being electrochemically degraded. In some instances, the delivery component 310 may be a polymer network/matrix, such as, for example, a hydrogel, which oxidatively breaks down due to the voltage at the electrode. As the polymer becomes soluble, the polymer and the supplemental agent are released from the electrode. The degradative network/matrix may facilitate quick and improved release of all agent from the electrode. In other embodiments, the polymer may be a hydrogel which swells and releases the agent so as to be delivered to the target site. Still, in other embodiments, the delivery component may include a polymer or sponge-type material capable of being saturated with a charged agent. In some cases the degradable polymer may also be entrained within a semipermeable membrane to facilitate maintaining the degradable polymer within close proximity of the electrode and lending mechanical stability to the materials.

In one particular embodiment, a conducting wire comprised of platinum, silver, or silver chloride may be thread alongside the needle and implanted for a short period of time. The conducting wire may be coated with a membrane made up of a hydrogel encapsulating agents such as anti-inflammatory drugs or chemotherapy agents. The counter electrode will be attached to the skin as a conducting pad or as a conducting surface on a Foley catheter. An electric potential may then be applied between the wire and conducting pad or Foley catheter. A power system allows for the control of the current and electric potential of the electrodes. This will enhance the transport of charged agent within the tissue.

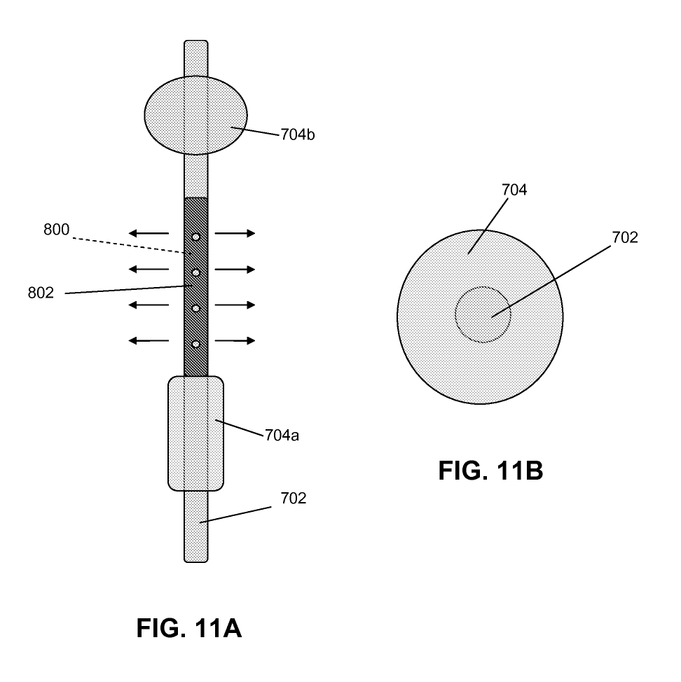

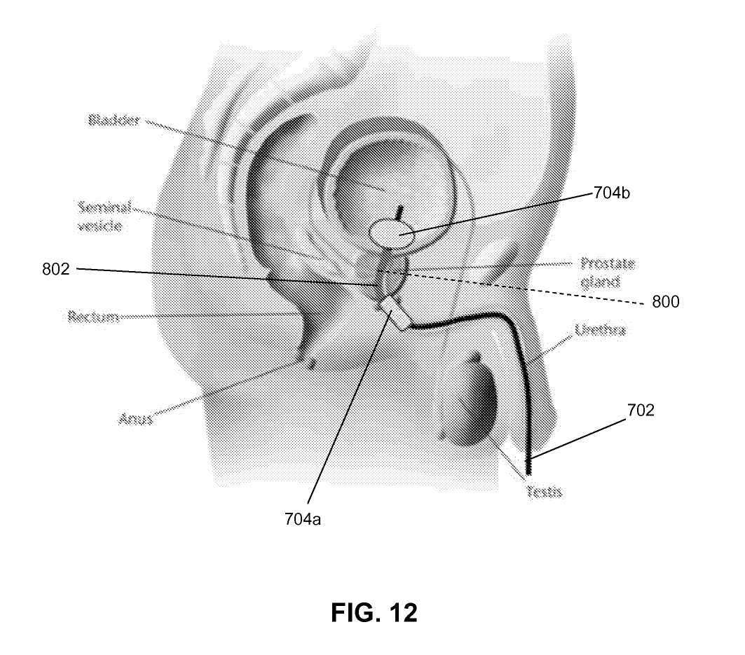

In other instances, the delivery system may include a modified catheter balloon apparatus, which can be used in conjunction with existing catheters, and may be used to encapsulate a degradable delivery component, as shown in FIGS. 11 and 12. The term catheter as used in the present application is intended to broadly include any medical device designed for insertion into a body passageway to permit injection or withdrawal of fluids, to keep a passage open or for any other purpose. In some instances, the term agent refers to a particle that contains a therapeutic. In some instances, the term agent refers to a therapeutic. A therapeutic can include a small molecule, biologic, or other substances utilized for the treatment or detection of disease. For example, the agent can be a device that collects in a tumor bed to interact with tissue.

The delivery system of the present invention has applicability for treating tissue and organ systems and, further, has applicability with any body passageway including, among others, blood vessels, tubular structures of the urinary, genitourinary, and intestinal tracts, the trachea and the like, and may be used to treat, for example, renal disease, uterine fibroids, urinary incontinence, erectile dysfunction, colorectal disease and inner and outer ear infections. One particular application of the delivery apparatus may include the delivery of therapeutic agents to the prostate gland, as illustrated in FIG. 12.

FIGS. 11 and 12 illustrate a delivery system which may deliver agents iontophoretically to target sites for localized treatment. Iontophoresis technology is known in the art and is commonly used in transdermal drug delivery. In general, iontophoresis technology uses an electrical potential or current across a semipermeable barrier to drive ionic fixatives or drugs or drag nonionic fixatives or drugs in an ionic solution. Iontophoresis facilitates both transport of the fixative or drug across the selectively permeable membrane and enhances tissue penetration. In the application of iontophoresis, two electrodes, one on each side of the barrier, are utilized to develop the required potential or current flow. In particular, one electrode may be located inside of the catheter in opposed relation to the drug delivery wall of the catheter while the other electrode may be located at a remote site on a patient's skin.

FIG. 11 illustrates one particular embodiment of the delivery system 700. In some instances, the delivery system 700 may include a flexible catheter body 702. The catheter 702 may be configured so as to be introduced the body through a guide catheter, or over a guide wire, or in another desirable manner. The catheter 702 may include an elongate portion with one or more electrodes 800 disposed thereon or otherwise engaged therewith. The catheter 702 is capable of insertion into an arterial vessel or other body passageway, such as, for example, the urethra, wherein the catheter 702 is extended through a vessel or other body passageway to be positioned proximate to the target site (i.e., the body tissue targeted for treatment or otherwise targeted for receipt of the agent).

The catheter may include a delivery component 802/drug reservoir disposed thereon and/or carried thereby. In some embodiments, the delivery component 802, carrying an ionically charged agent (or microparticles/nanoparticles carrying the agent), may traverse the interior of the catheter to reach the target site (e.g., urethral wall) so as to maintain the integrity of the delivery component 802. An electrical lead 24 may be provided so as to electrically connect the electrodes 800 to a power supply. A return electrode may be positioned, for example, on the surface of the patient's body and connected to the power supply by an electrical lead. In such instances, a voltage potential can be achieved between the electrodes such that the ionically charged agent is repelled from the electrode 70 and attracted to the return electrode to promote deep penetration of the agent into the body tissue. In some instances, the return electrode may have pressure-sensitive adhesive backing and low impedances at the skin to electrode interface. The electrode materials may minimize undesired oxidative/reductive reactions or production of competitive ions during the iontophoresis. For example, electrode materials may include platinum or any other suitable materials or, in other instances, silver for anodal electrodes and silver/silver chloride for cathodal electrodes.

The delivery component 802/drug reservoir may, in some instances, be constructed of a degradable structure capable of being electrochemically degraded. In some instances, the delivery component 802 may be a polymer network/matrix, such as, for example, a hydrogel, which oxidatively breaks down due to the voltage at the electrode. As the polymer becomes soluble, the polymer and the agent are released from the anode. The degradative network/matrix may facilitate quick and improved release of all agent from the electrode. In other embodiments, the polymer may be a hydrogel which swells and releases the agent so as to be delivered to the target site. Still, in other embodiments, the delivery component may include a polymer or sponge-type material capable of being saturated with a charged agent. In some cases the degradable polymer may also be entrained within a semipermeable membrane to facilitate maintaining the degradable polymer within close proximity of the electrode and lending mechanical stability to the materials.

FIG. 12 illustrates embodiments of the present invention which are provided to deliver an agent to a localized area of internal body tissue. As such, in some embodiments, the delivery system 700 may include a flexible catheter (e.g., a Foley catheter) 702 connected to one or more expandable components 704 and having an electrode 804/delivery component 802 operably engaged therewith. For example, the delivery system 700 may include a double balloon component 704 (e.g., a double Foley catheter), as schematically shown in FIGS. 11A and 12, which illustrates the balloon components 704 in an inflated/expanded state. In some instances, the catheter 702 may include a guide wire for positioning the catheter 702 near the target site, wherein a balloon lumen or passageway extends along the catheter 702 to facilitate inflation and deflation of the balloon components 704.

In one particular embodiment, as illustrated in FIG. 12, the delivery system 700 may comprise balloon components 704a and 704b that are provided in an inflated state within the urethra and the bladder of a patient. The catheter 702 may be advanced along the urethra to the desired position or site for treating the prostate gland. The balloon components 704a, 704b are then inflated by introducing an inflation fluid through the balloon lumen into the interior chambers of the balloon components 704a, 704b. In this manner, the electrode 800 and delivery component 802 may be maintained in a desired position (i.e., proximate to the prostate gland). As such, the embodiment illustrated in FIG. 12 may utilize iontophoresis to assist in driving a drug agent into the prostate gland. The electrode 800/delivery component 802 may be located on or within the catheter body 702 while the corresponding electrode, the body surface electrode, is located on the body surface (i.e., a patch applied to the patient's skin) or within the body of the patient. An electrical current is produced between the electrodes by an external power source, thereby creating a net flow of current between the electrodes. As previously described, the current flow may cause degradation of the delivery component 802 so as to facilitate the controllable release of the drug agent carried therewith. The released agent can diffuse into the prostate gland. In some instances, the net current flow drives or drags the agent, now released/deployed, within the prostate gland.

As such, embodiments of the present invention may include an iontophoretic delivery system (e.g., Foley catheter) for specific application in urological diseases and prostate cancer for patients undergoing brachytherapy procedures. The Foley catheter delivery system may contain an electrode that can deliver agents such as specific anti-inflammatory drugs and smooth muscle relaxants through the urethral wall. The Foley catheter delivery system may contain single or multiple source electrodes located on the catheter tube, wherein the counter electrode may be located on skin or in the prostate. The agent (e.g., drug) may be administered through a lumen of the Foley catheter and expelled around the site of the electrode. The electrode material may be composed of platinum, silver, or silver chloride, which is determined by the charge on the agent that is selected for delivery and the charge on the electrode. According to other embodiments, a double balloon Foley catheter delivery system may be used to minimize drug exposure and allow for the containment of the solubilized drug within a drug reservoir. According to another embodiment, a hydrogel surrounding an electrode on the body of the Foley catheter and carrying the agent may be used. In other embodiments, a drug-hydrogel coated balloon stent may be expanded from the Foley catheter, with a conducting wire connected to the stent. The stent may be retractable and can allow for transport of agent from the stent into the surrounding tissue.

The following examples are presented by way of illustration, not by way of limitation.

### Experimental

One embodiment of the present invention provides the ability to incorporate nanoparticle drug vehicles into prostate brachytherapy to improve the treament's therapeutic ratio. In general, any nanoparticle platform can be used for this application. In this particular formulation, Particle Replication In Non-wetting Templates (PRINT) technology was utilized to formulate dexamethasone loaded particles. In particular, poly(lactic-co-glycolic acid) (PLGA with molar ratio 85:15) was used as the base component for the particles. Different GRAS materials could be used for making the particles. In addition, spacers made of silo ether based biocompatible material were used.

The following examples provide proof of research and development in support of these exemplary goals.

### Results:

Particle Size—the size of the particles were measured with a ZetaPALS dynamic light scattering detector (Brookhaven Instruments Corporation, Holtsville, N.Y.). The particles showed a narrow distribution with mean size of 240±10 nm. The particles showed no aggregation or change in particle size even after storage for 7 days at −20° C.

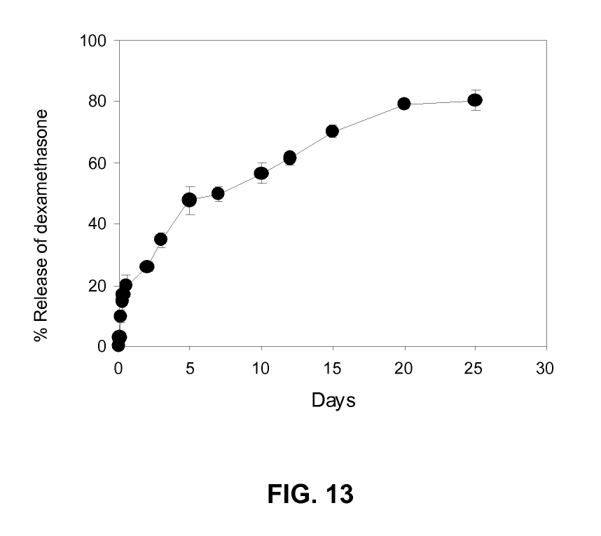

Release of dexamethasone from PRINT particles—the release of dexamethasone from PRINT particles in a phosphate buffer at 37° C. was studied. As shown in FIG. 13, the particles show stable retention of dexamethasone and controlled release over a period of 25 days. In particular, the particles show slow controlled release of about 30-40% within the first few days and long slow release thereafter over a period of time.

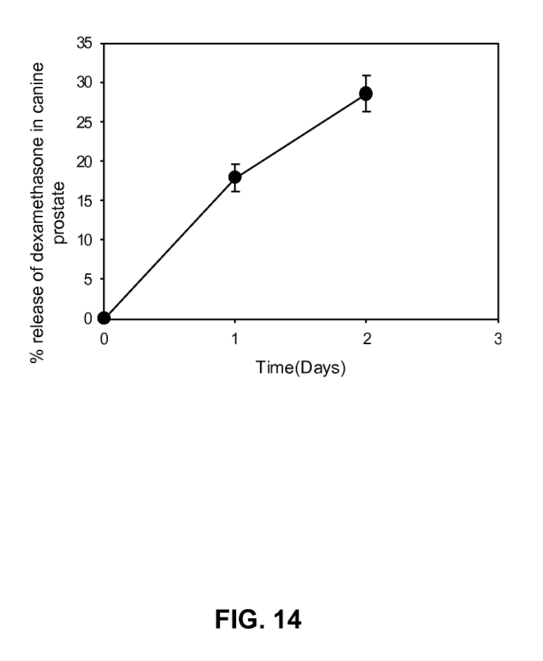

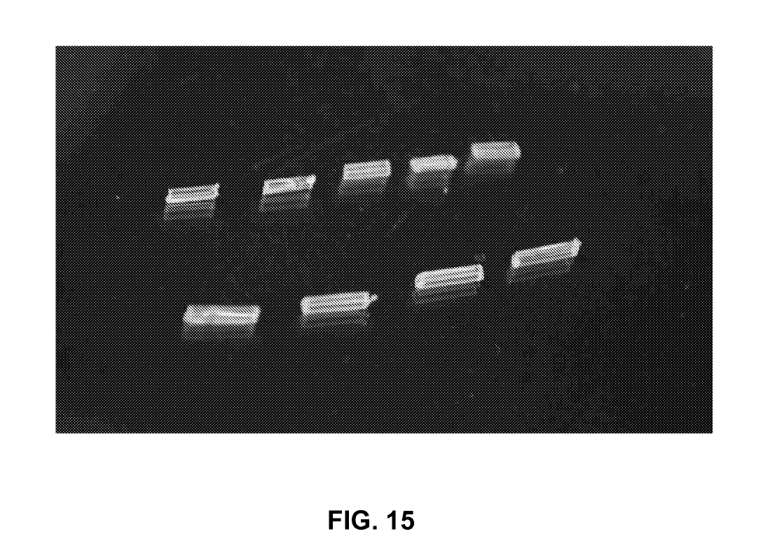

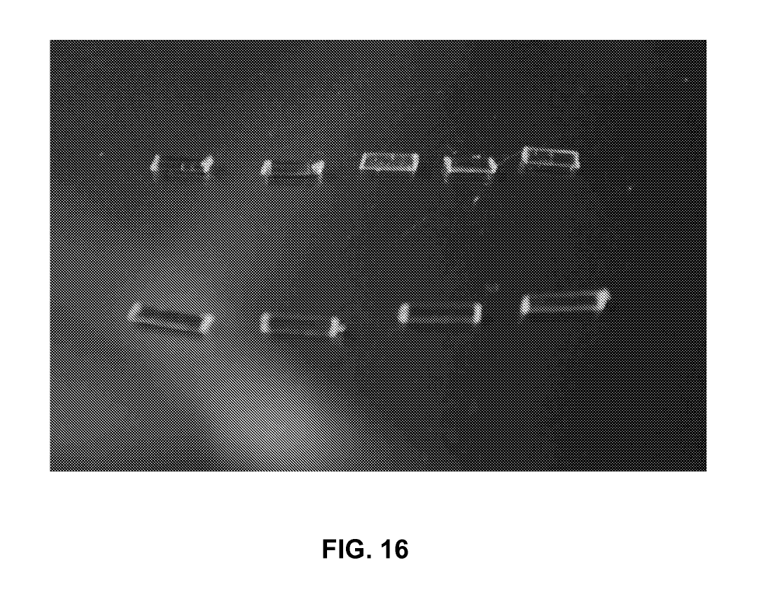

Release of dexamethasone in canine prostate—spacers made of silo ether based biocompatible material were also designed. In particular, the particles were suspended in a solution of 99% dimethyl silyl ether, 1% DEAP (photoinitiator). The material was photo cured for 3 minutes and spacers with dexamethasone loaded PRINT particles embedded in the matrix were obtained (see FIGS. 15 and 16). The release of dexamethasone from this system was studied ex vivo in canine prostate at 37° C. (shown in FIG. 14). The release studies were done by extracting dexamethasone from a small piece of prostate taken about 5 mm away from the point of insertion of the spacers. As desired, the spacers show slow controlled release over period of 2 days when the inflammation is expected.

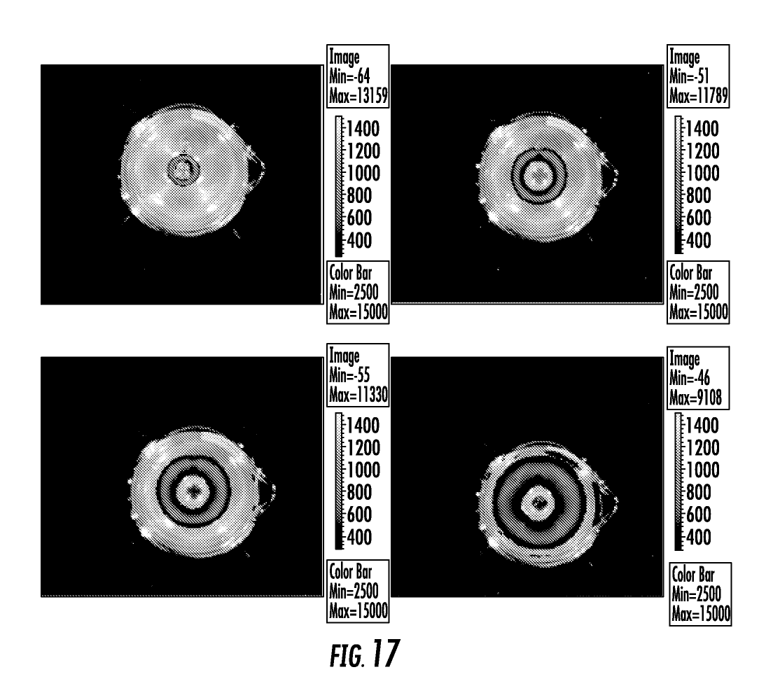

Release of encapsulated dye from PRINT particles—biodegradable spacers were embedded with PRINT particles made from dextrose, and a red fluorescent dye was encapsulated within the particles. The diffusion of the dye was observed within an aragose gel at 37° C. The dye diffused up to about 1 cm from the spacer within 6 hours as shown in FIG. 17.

Many modifications and other embodiments of the invention will come to mind to one skilled in the art to which this invention pertains having the benefit of the teachings presented in the foregoing description; and it will be apparent to those skilled in the art that variations and modifications of the present invention can be made without departing from the scope or spirit of the invention. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A delivery system adapted for implementation during a brachytherapy procedure, the delivery system comprising:
a plurality of brachytherapy seeds; and
a plurality of spacers, each spacer comprising a matrix material carrying at least one of a plurality of microparticles or nanoparticles, the at least one of the microparticles or nanoparticles carrying at least one agent and being at least one of biodegradable or biocompatible,
wherein the brachytherapy seeds and the spacers are configured to be delivered to a target site.

2. The delivery system of claim 1, wherein each spacer comprises a matrix material mixed with at least one of a plurality of microparticles or nanoparticles.

3. The delivery system of claim 1, wherein each spacer comprises a matrix material housing the at least one of the plurality of microparticles or nanoparticles.

4. The delivery system of claim 1, wherein the agent comprises at least one of anti-inflammatory agents, anti-androgen agents, chemotherapy agents, agents that improve urination, genetic agents, siRNA, imaging agents, protein agents, biologics, molecular targeted agents against cancer, or alpha blocker agents.

5. The delivery system of claim 4, wherein the agent comprises an anti-inflammatory agent comprising at least one dexamethasone, ibuprofen, prednisone, betamethasone, or glucocorticoid.

6. The delivery system of claim 4, wherein the agent comprises an agent that improves urination comprising at least one of terazosin or tamsulosin.

7. The delivery system of claim 4, wherein the agent comprises a chemotherapy agent comprising at least one of docetaxel, paclitaxel, cisplatin, gemcitabine, or doxorubicin.

8. The delivery system of claim 4, wherein the agent comprises an anti-androgen agent comprising at least one of bicalutamide, flutamide, or nilutamide.

9. The delivery system of claim 1, wherein the agent is encapsulated by at least one of the nanoparticles or microparticles.

10. The delivery system of claim 1, wherein at least a portion of the at least one of the microparticles or nanoparticles are biodegradable.

11. The delivery system of claim 1, wherein the at least one of the microparticles or nanoparticles are about 10 nm to 500 μm in diameter.

12. The delivery system of claim 1, wherein the at least one of the microparticles or nanoparticles are about 230-250 nm in diameter.

13. The delivery system of claim 1, wherein the at least one of the microparticles or nanoparticles are fabricated using a soft or an imprint lithography technique.

14. The delivery system of claim 1, wherein each of the plurality of brachytherapy seeds comprises a radioactive isotope.

15. The delivery system of claim 1, wherein the matrix material comprises at least one of a plastic, a synthetic biodegradable polymer, or a natural polysacchride.

16. The delivery system of claim 1, wherein at least a portion of the matrix material comprises a biodegradable material.

17. The delivery system of claim 1, wherein the at least one of the microparticles or nanoparticles comprises at least one of poly(lactic-co-glycolic acid), polylactic acid, polyglycolic acid, chitosan, lipids, poly(R-aminoester), polyethylenes, polycarbonates, polyanhydrides, polyhydroxyacids, polypropylfumerates, polycaprolactones, polyamids, polyacetals, polyethers, polyesters, polyorthoesters, polycyanoacrylates, polyvinyl alcohols, polyurethanes, polyphosphazenes, polyacrylates, polymethacrylates, polyureas, polystyrenes, polyamines, proteins, lipids, surfactants, carbohydrates, small molecules, or polynucleotides.

18. The delivery system of claim 1, wherein the plurality of brachytherapy seeds and spacers are arranged in an alternating pattern such that each seed is separated by a spacer.

19. The delivery system of claim 1, wherein the plurality of brachytherapy seeds and spacers are coupled to one another.

20. The delivery system of claim 1, further comprising an insertion device configured to receive each of the plurality of brachytherapy seeds and the plurality of spacers and deliver the seeds and spacers to the target site.

21. The delivery system of claim 20, wherein the insertion device comprises at least one needle.

22. The delivery system of claim 1, wherein the agent is configured to be gradually released over time.

23. A method for co-delivery of an agent during a brachytherapy procedure, the method comprising:
delivering a plurality of brachytherapy seeds proximate to a target site; and
delivering a plurality of spacers proximate to the target site, each spacer comprising a matrix material carried by at least one of a plurality of microparticles or nanoparticles, the at least one of the microparticles or nanoparticles carrying at least one agent and being at least one of biodegradable or biocompatible.

24. The method of claim 23, wherein delivering the plurality of brachytherapy seeds and the plurality of spacers comprises collectively delivering the brachytherapy seeds and the spacers.

25. The method of claim 23, wherein delivering the plurality of brachytherapy seeds and the plurality of spacers comprises independently delivering the brachytherapy seeds and the spacers.

26. The method of claim 23, wherein delivering the plurality of brachytherapy seeds and the plurality of spacers comprises delivering the brachytherapy seeds and the spacers while coupled to one another.

27. The method of claim 23, wherein delivering the plurality of brachytherapy seeds and the plurality of spacers comprises delivering the brachytherapy seeds and the spacers in an alternating pattern.

28. The method of claim 23, wherein delivering the plurality of brachytherapy seeds and the plurality of spacers comprises delivering the brachytherapy seeds and the spacers with an insertion device.

29. The method of claim 23, wherein delivering the plurality of spacers comprises delivering the spacers such that the agent is gradually released over time.

30. The method of claim 23, wherein delivering the plurality of brachytherapy seeds and the plurality of spacers comprises delivering the brachytherapy seeds and the spacers into a prostate gland.
